(19) United States
(12) Patent Application Publication
(10) Pub. No.: US 2021/0205354 A1
(43) Pub. Date: Jul. 8, 2021

(54) ANTI-INFLAMMATORY COMPOSITION COMPRISING HYDROGEN ANIONS

(71) Applicant: VOVI C&E CO.,LTD., Seoul (KR)

(72) Inventors: Tae Ho CHOI, Seoul (KR); Young Hyuk JUNG, Gyeonggi-do (KR); Young Yong IN, Jeollabuk-do (KR); Jin Sol CHOI, Gangwon-do (KR); Bo Mi KIM, Gyeonggi-do (KR)

(21) Appl. No.: 17/059,921

(22) PCT Filed: Oct. 18, 2018
(86) PCT No.: PCT/KR2018/012325

§ 371 Date: Nov. 30, 2020

(51) Int. Cl. A61K 33/10; A61K 33/08

(52) U.S. Cl. CPC A61K 33/10; A61K 33/08

(57) ABSTRACT

An anti-inflammatory composition according to an embodiment includes natural organic calcium carbonate, and thus provides the advantages that the composition can be consumed as food, is completely harmless to the human body, and can significantly treat or alleviate inflammations in human and animal cells. Also, the anti-inflammatory composition can promote metabolisms of cells and eliminate active oxygen in the body while treating inflammations. Since the anti-inflammatory composition can be preserved easily because it is not sensitive to the surrounding environment and is resistant to degradation even when exposed to the atmosphere, the anti-inflammatory composition can be added to various beverages or food materials. Furthermore, when the anti-inflammatory composition is added to food materials, a high degree of freshness of food can be preserved for a long time by preventing the oxidation thereof, and also has the effect of restoring oxidized skin and damaged skin through reduction.

### TECHNICAL FIELD

The present disclosure relates to an anti-inflammatory composition having hydrogen anions, more particularly to a nonsteroidal anti-inflammatory composition which uses natural organic calcium carbonate, and thus provides the advantages that it can be consumed as food, is completely harmless to the human body, and can significantly treat or alleviate inflammations in human and animal cells.

### BACKGROUND ART

Most of the currently used anti-inflammatory drugs are steroid-based compounds including glucocorticoids. These drugs are called corticosteroids and serve to reduce swelling and inflammation of cells by binding to the glucocorticoid receptors of the cells.

However, the steroid-based anti-inflammatory drugs cause various adverse effects in cells. For this reason, nonsteroidal anti-inflammatory drugs (NSAIDs) have been developed as described in the patent document 1.

The nonsteroidal anti-inflammatory drugs relieve pain by inhibiting the cyclooxygenase (COX) enzyme. The COX enzyme synthesizes prostaglandins, which cause inflammations. All NSAIDs can relieve pain because they inhibit the synthesis of prostaglandins.

Typical examples of the NSAIDs include aspirin, ibuprofen, naproxen, etc. COX-specific drugs that emerged later are distinguished from classic NSAIDs although the mechanism action is similar to that of the NSAIDs. The classic NSAIDs indiscriminately inhibit COX1 which is expressed at all times and COX2 which occurs only in inflammations.

Therefore, the NSAIDs do not act on inflammations only but have the risk of causing various adverse effects. One of the representative adverse effects is the gastrointestinal trouble of aspirin. Although COX2-specific inhibitors such as celecoxib, rofecoxib, etc. have emerged to solve this problem, they do not provide a complete solution.

In addition, long-term use of the NSAIDs may cause gastrointestinal troubles and gastric ulcers, aggravate asthma and cause kidney problems. In addition, overdoses of acetaminophen can cause liver damage.

Accordingly, there is an acute need of the development of an anti-inflammatory drug which uses a natural organic material; and thus can be consumed as food with no harm to the human body. The applicant incidentally found out that a hydrogen anion-containing material of Korean Patent No, 10-1405431 (patent document 2) has an anti-inflammatory property of treating cellular inflammation. The present disclosure is based on the incidental finding.

### REFERENCES OF RELATED ART

### Patent Documents

Patent document 1: Korean Patent Publication No. 10-2007-0071534 (Jul. 4, 2007).

Patent document 2: Korean Patent Registration No. 10-1405431 (Jun. 11, 2014).

### DISCLOSURE

### Technical Problem

The present disclosure is directed to providing an anti-inflammatory composition that can be consumed as food with no harm to the human body.

### Technical Solution

In an exemplary embodiment of the present disclosure, an anti-inflammatory composition containing hydrogen anions is prepared through: a first firing step of preparing a first firing product by firing an alkaline earth metal compound containing one or more organic calcium carbonate selected from a group consisting of eggshell calcium, pearl calcium, shell calcium and seaweed calcium; a second firing step of preparing a second firing product by firing an alkaline earth metal oxide containing the first firing product under hydrogen atmosphere; and a step of mixing the second firing product with an organic add.

In another exemplary embodiment of the present disclosure, in the first firing step, the alkaline earth metal compound may be fired at 300-1000° C. for 2-10 hours.

In another exemplary embodiment of the present disclosure, the alkaline earth metal compound may further include a beryllium compound, a magnesium compound, a barium compound or a mixture thereof.

In another exemplary embodiment of the present disclosure, beryllium oxide, magnesium oxide, barium oxide or a mixture thereof may be added in the second firing step.

In another exemplary embodiment of the present disclosure, the alkaline earth metal oxide may include 1-60 mol of beryllium oxide, magnesium oxide, barium oxide or a mixture thereof based on 100 mol of calcium oxide.

In another exemplary embodiment of the present disclosure, in the second firing step, the alkaline earth metal oxide may be fired at 300-1000° C. for 2-10 hours under hydrogen atmosphere.

In another exemplary embodiment of the present disclosure, the second firing product may be mixed with 0.5-10 mol of an organic acid based on 100 mol of calcium ions included in the second firing product.

The anti-inflammatory composition according to the present disclosure may have a pH (hydrogen ion concentration) of 6-12 immediately after being dissolved in water; or may have an oxidation/reduction potential of −0.1 mV to −900 mV or may have a reducing power duration of 1-150 hours immediately after being dissolved in water.

### Advantageous Effects

An anti-inflammatory composition according to the present disclosure, which uses natural organic calcium carbonate, has advantages that it can be consumed as food with no harm to the human body and can treat or alleviate the inflammations of human and animal cells remarkably. In addition, the anti-inflammatory composition according to the present disclosure can promote metabolisms of cells and eliminate active oxygen in the body while treating inflammations. In addition, since the anti-inflammatory composition according to the present disclosure can be preserved easily because it is not sensitive to the surrounding environment, such as changes in temperature, and is resistant to degradation even when exposed to the atmosphere, the anti-inflammatory composition can be added to various beverages or various food materials and can be used as food. In addition, the anti-inflammatory composition can be added to food materials such as fruits, vegetables, fish and meat to allow the food materials to preserve a high degree of freshness for a long time by preventing the oxidation thereof, and also has the effect of restoring oxidized skin and damaged skin through reduction.

### BRIEF DESCRIPTION OF THE DRAWINGS

FIG. 1 is an image of an aqueous solution wherein an anti-inflammatory composition of the present disclosure is dissolved.

FIG. 2 shows a result of testing the cytotoxicity of an anti-inflammatory composition of the present disclosure.

FIG. 3 shows a result of investigating the anti-inflammatory effect of an anti-inflammatory composition of the present disclosure.

### BEST MODE

Hereinafter, exemplary embodiments of the present disclosure will be described in detail so that those of ordinary skill in the art to which the present disclosure belongs can easily carry out the present disclosure. However, the present disclosure can be embodied into various different forms and the present disclosure is not limited to the exemplary embodiments described herein.

The adverse effects of steroidal anti-inflammatory drugs are widely known. Although nonsteroidal anti-inflammatory drugs (NSAIDs) have been developed to make up for this problem, long-term use of the NSAIDs can cause gastrointestinal troubles and gastric ulcers, aggravate asthma and cause kidney problems.

Therefore, the applicant has developed an anti-inflammatory drug which uses natural organic material and thus can be consumed as food with no harm to the human body. This development is based on the hydrogen anion-containing material of Korean Patent No. 10-1405431 which has been filed and registered by the applicant.

The applicant incidentally found out that the hydrogen anion-containing material of Korean Patent No. 10-1405431 has an anti-inflammatory property of treating inflammations in human and animal cells. The present disclosure is based on the incidental finding.

Recently, various types of products based on the antioxidative action of anions are available on the market. Examples include ion generators capable of purifying indoor air and negative ion products capable of removing lime smell of tap water and mineralizing the same.

In addition, negative ion food products are available on the market, beyond the level of simply improving the living environment. However, the negative ion products available on the market fail to promote antioxidative actions sufficiently in the body due to poor solubility for water. In addition, the negative ion products are difficult to be stored for a long time and wheat flour, etc. used to make the negative ion products cause many adverse effects. Therefore, the applicant has developed an edible negative ion product, which has been registered as Korean Patent No. 10-1405431, For details, refer to Korean Patent No. 10-1405431.

An anti-inflammatory composition according to an exemplary embodiment of the present disclosure is prepared through a method including: a first firing step of preparing a first firing product by firing an alkaline earth metal compound containing organic calcium carbonate; a second firing step of preparing a second firing product by firing an alkaline earth metal oxide containing the first firing product; and a step of mixing the second firing product with an organic acid.

The organic calcium carbonate is an organic material containing calcium carbonate and is distinguished from calcium carbonate obtained from inorganic materials such as ores. The organic calcium carbonate contains a very small amount of heavy metals or contains no heavy metal at all when compared with calcium carbonate obtained from inorganic materials such as ores. In addition, the organic calcium carbonate is rich in minerals when compared with calcium carbonate obtained from inorganic materials such as ores. Accordingly, the organic calcium carbonate is very suitable to prepare an edible hydrogen anion-containing material.

As the organic calcium carbonate, for example, one or more selected from a group consisting of eggshell calcium, pearl calcium, shell calcium and seaweed calcium may be used.

The eggshell calcium may be obtained from egg shell, etc., the shell calcium may be obtained from oyster, clam, abalone, fish bone, etc., and the seaweed calcium may be obtained from starfish, coral, etc. When considering the easiness of procurement, eggshell calcium, shell calcium or a mixture thereof may be used as the organic calcium carbonate.

Methods for preparing a hydrogen anion-containing material may be largely classified into two types. As a first exemplary embodiment, organic calcium carbonate as an alkaline earth metal compound is physically mixed with a compound containing an alkaline earth metal which is not calcium. In a second exemplary embodiment, organic calcium carbonate is used as an alkaline earth metal compound.

In the first exemplary embodiment, the alkaline earth metal compound may further contain, in addition to the organic calcium carbonate, a beryllium compound, a magnesium compound, a barium compound or a mixture thereof. Since the compound further containing an alkaline earth metal other than calcium affects the reducing power of the prepared hydrogen anion-containing material, the reducing power of the hydrogen anion-containing material may be controlled by selecting a suitable compound.

In an exemplary embodiment, the alkaline earth metal compound may further contain a magnesium compound in addition to the organic calcium carbonate. The magnesium compound may effectively control the reducing power of the hydrogen anion-containing material. In addition, it can help excretion of sodium through interaction with calcium, help blood flow by promoting the production of nitric oxide, and alleviate hangover and fatigue by reducing acetaldehyde, etc.

The magnesium compound is not specially limited as long as it can produce magnesium oxide through the first firing step. Examples of the magnesium compound may include magnesium (Mg), magnesium chloride (MgCl₂), magnesium stearate (Mg(C₁₈H₃₅O₂)₂), magnesium carbonate (MgCO₃), magnesium sulfate (MgSO₄), magnesium hydroxide (Mg(OH)₂), trimagnesium phosphate (Mg₃(PO₄)₂), dimagnesium phosphate (MgHPO₄), magnesium silicate (MgSiO₃), magnesium gluconate (C₁₂H₂₂MgO₁₄), magnesium L-lactate (Mg(C₃H₅O₃)₂.2H₂O), magnesium oxide (MgO) or a mixture thereof.

The compound containing an alkaline earth metal which is not calcium may be used at an appropriate content in consideration of the desired reducing power of the hydrogen anion-containing material, duration of the reducing power and/or concentration of hydrogen anion. In an exemplary embodiment, the compound may be contained in an amount of 1-60 mol, 1.6-57 mol, 1.6-50 mol, 1.6-40 mol, 1.6-30 mol, 5-20 mol or 5-15 mol based on 100 mol of the calcium carbonate contained in the organic calcium carbonate. If the content of the compound is less than the above-described range, the reducing power of the hydrogen anion-containing material may be too weak or the reducing power duration may be too short. And, if it exceeds the above-described range, the reducing power of the hydrogen anion-containing material may be too strong for consumption. In this case, the hydrogen anion-containing material may interfere with the metabolism in the body.

In an exemplary embodiment, when magnesium compound is used as the compound containing an alkaline earth metal which is not calcium, the magnesium compound may be used in an amount of 0.5-20 parts by weight based on 80-99.5 parts by weight of the calcium carbonate contained in the organic calcium carbonate. Through this, a hydrogen anion-containing material exhibiting the above-described effect may be provided.

In the first firing step, the alkaline earth metal compound may be fired at about 300-1000° C., about 400-900° C., about 500-800° C., about 600-800° C., about 650-800° C., about 600-750° C. or about 650-750° C. And, the firing of the alkaline earth metal compound may be performed for about 2 hours or longer, about 3 hours or longer or about 4 hours or longer. The upper limit of the firing time is not particularly limited. For example, it may be controlled to 10 hours or shorter. If the firing is performed below the above-described temperature range and shorter than the above-described temperature range, the alkaline earth metal compound may not be oxidized as desired. And, if the firing is performed higher than the above-described temperature range and longer than the above-described temperature range, a large amount of energy will be wasted.

The first firing step may be performed in the atmosphere, although not being specially limited thereto.

When preparing the hydrogen anion-containing material, a step of cooling the first firing product may be further included after the first firing step. The first firing product may be cooled, for example, by natural cooling, although not being specially limited thereto. The natural cooling may be performed by leaving the first firing product held in a container alone until room temperature or a target temperature is reached. In the present disclosure, the room temperature means an ambient temperature in a state without heating or cooling.

The first firing product that has passed through the first firing step according to the first exemplary embodiment may contain calcium oxide (CaO) obtained from the organic calcium carbonate and beryllium oxide (BeO), magnesium oxide (MgO), barium oxide (BaO) or a mixture thereof, obtained from the compound containing an alkaline earth metal which is not calcium.

In an exemplary embodiment, when a magnesium compound is used as the compound containing an alkaline earth metal which is not calcium, the first firing product may contain calcium oxide and magnesium oxide.

In contrast, the first firing product that has passed through the first firing step according to the second exemplary embodiment contains calcium oxide (CaO) obtained from the organic calcium carbonate. Accordingly, in this case, an oxide containing an alkaline earth metal which is not calcium may be added in the second firing step, so as to achieve the same composition as the first firing product prepared according to the first exemplary embodiment. The oxide containing an alkaline earth metal which is not calcium may be beryllium oxide (BeO), magnesium oxide (MgO), barium oxide (BaO) or a mixture thereof.

In an exemplary embodiment, the oxide containing an alkaline earth metal which is not calcium may be magnesium oxide. That is to say, in the second exemplary embodiment, the first firing product and magnesium oxide as the alkaline earth metal oxide may be used. Through this, the alkaline earth metal oxide may have the same composition for both the first exemplary embodiment and the second exemplary embodiment.

The alkaline earth metal oxide may contain 1-60 mol, 1.6-57 mol, 1.6-50 mol, 1.6-40 mol, 1.6-30 mol, 5-20 mol or 5-15 mol of the oxide containing an alkaline earth metal which is not calcium based on 100 mol of calcium oxide. Through this, a hydrogen anion-containing material with appropriate reducing power may be prepared.

In addition, the first firing product according to the first exemplary embodiment and the second exemplary embodiment may further contains various minerals derived from the organic calcium carbonate, in addition to the components described above. As a result, the final product, or the hydrogen anion-containing material, may also contain various minerals derived from the organic calcium carbonate.

In the second firing step, the alkaline earth metal oxide may be fired at about 300-1000° C., about 300-900° C., about 400-900° C., about 400-800° C., about 500-800° C., about 500-700° C. or about 600-700° C. In addition, the alkaline earth metal oxide may be fired for about 2 hours or longer, about 3 hours or longer or about 4 hours or longer. The upper limit of the firing time is not particularly limited. For example, it may be controlled to 10 hours or shorter. If the firing is performed below the above-described temperature range and shorter than the above-described temperature range, the alkaline earth metal oxide may not be reduced as desired. And, if the firing is performed higher than the above-described temperature range and longer than the above-described temperature range, a large amount of energy will be wasted.

The second firing step may be performed under hydrogen atmosphere. Accordingly, hydrogen gas may be supplied while the alkaline earth metal oxide is fired. The hydrogen gas may be injected either alone or together with another carrier gas. As the carrier gas, any commonly used gas with no reactivity in the firing temperature range described above may be used without special limitation. Examples of the carrier gas may include nitrogen, helium, argon, etc.

The method for preparing an anti-inflammatory composition according to the present disclosure may further include a step of cooling the second firing product after the second firing step. A method for cooling the second firing product is not specially limited, and the method used to cool the first firing product may be used.

A method for preparing the hydrogen anion-containing material includes, after the second firing step, mixing the second firing product with an organic acid.

The second firing product contains calcium hydride (CaH₂) and a hydride of an alkaline earth metal which is not calcium (MH₂, M=Be, Mg or Ba). In the present disclosure, for distinction of the ┌second firing product┘ and the ┌second firing product mixed with an organic acid┘, the former is referred to as a second firing product and the latter is referred to as a hydrogen anion-containing material.

The mixing of the second firing product with the organic acid may be performed by physical mixing by adding the organic acid to a container holding the second firing product.

As the organic acid, for example, one or more selected from a group consisting of formic acid, acetic acid, citric acid, lactic acid, tartaric acid and succinic acid may be used. In an exemplary embodiment, an organic acid which is solid at room temperature may be used to prepare a hydrogen anion-containing material in the form of powder. Accordingly, citric acid, lactic acid, tartaric acid, succinic acid or a mixture thereof may be used as the organic acid.

In the step of mixing the second firing product with the organic acid, 0.5-10 mol, 0.88-10 mol, 1-9 mol, 2-8 mol, 3-8 mol, 3-7 mol or 3-6 mol of the organic acid may be mixed based on 100 mol of calcium ion contained in the second firing product. Within this range, the hydrogen anion-containing material has reducing power and duration of the reducing power appropriate for inducing suitable antioxidative action. The duration of the reducing power refers to the time during which an aqueous solution wherein the hydrogen anion-containing material is desired exhibits a negative oxidation/reduction potential (ORP) value.

In addition, the hydrogen anion-containing material containing the organic acid of the above-described content may have a pH which is suitable in the body. In an exemplary embodiment, if the pH measured by adding 400 mg of the second firing product to 300 mL of water is about 10-12, the pH value may be decreased to about 6-11 by mixing the organic acid with the second firing product in the range described above. Accordingly, a hydrogen anion-containing material which exerts the effect described by the present disclosure without burden to the body may be provided.

The anti-inflammatory composition according to the present disclosure may have a pH (hydrogen ion concentration) of 6-12, 7-12, 8-12, 8-11 or 9-11 immediately after being dissolved in water. In addition, the hydrogen anion-containing material may have an appropriate reducing power with an oxidation/reduction potential of −0.1 mV to −900 mV, −0.1 mV to −800 mV, −0.1 mV to −700 mV, −0.1 mV to −600 mV −0.1 mV to −500 mV, −10 mV to −450 mV, −100 mV to −400 mV, −150 mV to −300 mV, −180 mV to −250 mV or −190 mV to −230 mV immediately after being dissolved in water. In addition, the duration of the reducing power may be 1-150 hours, 5-120 hours, 10-100 hours, 30-100 hours, 50-100 hours or 70-100 hours. These physical properties are based on measurement by adding 400 mg of the hydrogen anion-containing material to 300 mL of water.

In an exemplary embodiment, the anti-inflammatory composition prepared according to the present disclosure may have a pH of 9-11 immediately after being dissolved in water. In addition, the anti-inflammatory composition may have an oxidation/reduction potential of −190 mV to −230 mV immediately after being dissolved in water. And, the duration of the reducing power may be about 50-100 hours. The hydrogen anion-containing material having such physical properties can be consumed without burden to the body, can promote metabolisms of cells and effectively eliminate active oxygen in the body. The anti-inflammatory composition having such physical properties can be prepared using an alkaline earth metal compound or oxide of an appropriate content.

The anti-inflammatory composition prepared according to the method described above is not sensitive to the change in surrounding environment.

The anti-inflammatory composition prepared according to the method described above can be added, for example, to fruits, vegetables, fish and meat to prevent oxidation.

The anti-inflammatory composition prepared according to the method described above not only has the anti-inflammatory activity of treating and alleviating inflammations of human and animal cells but also can repair oxidized skin and damaged skin through reductive actions. In addition, the anti-inflammatory composition can be consumed as food and may be used widely in cosmetic materials, health foods, hangover-relieving beverages, etc.

### PREPARATION EXAMPLES: PREPARATION OF ANTI-INFLAMMATORY COMPOSITION

### Example 1

100 g of washed oyster shell was fired at 700° C. for 4 hours under air atmosphere to obtain a first firing product. The first firing product contained 56.08 g of calcium oxide.

Subsequently, 4.45 g of magnesium oxide (CaO:MgO=92.6 w:7.4 wt %=100 mol:11 mol) was added to the first firing product held in a reaction vessel. Then, the first firing product and an alkaline earth metal oxide containing magnesium oxide were fired at 650° C. for 4 hours or longer under hydrogen atmosphere to obtain a second firing product.

Subsequently, an anti-inflammatory composition in powder form was prepared by mixing the second firing product with citric acid at a ratio of 17.5 wt % based on the entire hydrogen anion-containing material.

### Example 2

An anti-inflammatory composition was prepared in the same manner as in Example 1, except that the content of magnesium oxide was changed to 0.66 g as described in Table 1.

### Example 3

An anti-inflammatory composition was prepared in the same manner as in Example 1, except that the content of magnesium oxide was changed to 9.53 g as described in Table 1.

### Example 4

An anti-inflammatory composition was prepared in the same manner as in Example 1, except that the content of magnesium oxide was changed to 22.95 g as described in Table 1.

### Example 5

An anti-inflammatory composition was prepared in the same manner as in Example 1, except that the content of citric acid mixed with the second firing product was changed to 12.5 wt % based on the entire hydrogen anion-containing material as described in Table 1.

### Example 6

An anti-inflammatory composition was prepared in the same manner as in Example 1, except that the content of citric acid mixed with the second firing product was changed to 25.0 wt % based on the entire hydrogen anion-containing material as described in Table 1.

TABLE 1

| | | | Second firing | Calcium |
|---|---|---|---|---|
| | | | product:organic | ion:organic |
| | CaO:MgO⁽¹⁾ | CaH₂:MgH₂⁽²⁾ | acid⁽³⁾ | acid⁽⁴⁾ |
| Example | 92.6 wt %:7.4 wt % | 93.54 wt %:6.46 wt % | 82.5 wt %:17.5 wt % | 100 mol:5 mol |
| 1 | 100 mol:11 mol | | | |
| Example | 98.84 wt %:1.16 wt % | 98.99 wt %:1.01 wt % | 82.5 wt %:17.5 wt % | 100 mol:4.7 mol |
| 2 | 100 mol:1.64 mol | | | |
| Example | 85.48 wt %:14.52 wt % | 87.12 wt %:12.88 wt % | 82.5 wt %:17.5 wt % | 100 mol:5.3 mol |
| 3 | 100 mol:23.64 mol | | | |
| Example | 70.96 wt %:29.04 wt % | 73.74 wt %:26.26 wt % | 82.5 wt %:17.5 wt % | 100 mol:6.3 mol |
| 4 | 100 mol:59.94 mol | | | |
| Example | 92.6 wt %:7.4 wt % | 93.54 wt %:6.46 wt % | 87.5 wt %:12.5 wt % | 100 mol:3.35 mol |
| 5 | 100 mol:11 mol | | | |
| Example | 92.6 wt %:7.4 wt % | 93.54 wt %:6.46 wt % | 75.0 wt %:25.0 wt % | 100 mol:7.8 mol |
| 6 | 100 mol:11 mol | | | |

⁽¹⁾Raito of calcium oxide and magnesium oxide in alkaline earth metal oxide,
⁽²⁾Raito of calcium hydride and magnesium hydride in second firing product,
⁽³⁾Raito of second firing product and organic acid in hydrogen anion-containing material,
⁽⁴⁾Raito of organic acid to calcium ion contained in second firing product

### Test Example: Measurement of Physical Properties of Anti-Inflammatory Composition

Change in oxidation/reduction potential (ORP), pH change and duration of reducing power were measured for the anti-inflammatory compositions prepared in the examples, and the turbidity of aqueous solutions wherein the anti-inflammatory compositions were dissolved were observed. In addition, the physical properties of the anti-inflammatory compositions of the examples were compared with tap water (Comparative Example 1) and a first firing product (Comparative Example 2: the first firing product of Example 1). The result is given in Table 2.

TABLE 2

| | Change in ORP (mV) and pH⁽¹⁾ | | | | | | | | Duration | Turbidity |
|---|---|---|---|---|---|---|---|---|---|---|
| | Time | | | | | | | | of reducing | of aqueous |
| | (hr) | Initial | 4 | 20 | 40 | 60 | 80 | 100 | power⁽²⁾ | solution⁽³⁾ |
| Comp. | ORP | 523 | 187 | 161 | 150 | 147 | 184 | 194 | — | — |
| Ex. 1 | pH | 8.1 | 8.2 | 8.4 | 8.5 | 8.4 | 8.5 | 8.6 | | |
| Comp. | ORP | 267 | 66 | 48 | 80 | 98 | 107 | 120 | — | Turbid |
| Ex. 2 | pH | 9.5 | 9.5 | 9.4 | 9.2 | 9.0 | 8.8 | 8.6 | | |
| Ex. 1 | ORP | −219 | −197 | −149 | −86 | −34 | 12 | 59 | 74 hr | Clear |
| | pH | 8.1 | 9.9 | 10.0 | 10.1 | 10.0 | 10.1 | 9.8 | | |
| Ex. 2 | ORP | −16 | −1 | 54 | 101 | 157 | 165 | 179 | 4 hr | Clear |
| | pH | 8.9 | 10.7 | 11.0 | 11.3 | 11.2 | 11.3 | 11.0 | | |
| Ex. 3 | ORP | −403 | −427 | −364 | −270 | −202 | −142 | −60 | 110 hr | Clear |
| | pH | 8.3 | 10.1 | 10.3 | 10.3 | 10.1 | 10.2 | 9.9 | | |
| Ex. 4 | ORP | −737 | −823 | −663 | −423 | −222 | −10 | 239 | 82 hr | Clear |
| | pH | 7.8 | 9.6 | 10.1 | 10.6 | 10.5 | 10.5 | 10.2 | | |
| Ex. 5 | ORP | −216 | −186 | −162 | −120 | −64 | 19 | 85 | 75 hr | Clear |
| | pH | 8.8 | 9.4 | 10.3 | 10.4 | 10.2 | 10.1 | 10.0 | | |
| Ex. 6 | ORP | −204 | −154 | −154 | −139 | −80 | 50 | 139 | 71 hr | Clear |
| | pH | 6.3 | 7.1 | 8.6 | 9.6 | 9.7 | 9.5 | 9.5 | | |

(1) Change in ORP (mV) and pH: After adding 300 mL of tap water to 8 beakers, nothing was added to one beaker (Comparative Example 1), 400 mg of the first firing product prepared in Example 1 was dissolved in another beaker (Comparative Example 2), and 400 mg the of anti-inflammatory compositions prepared in Examples 1-6 were dissolved in the remaining 6 beakers (Examples 1-6), respectively. For the tap water and the prepared aqueous solutions, ORP and pH were measured at the time when the first firing product or the hydrogen anion-containing material was dissolved (initial) and 4 hours, 20 hours, 40 hours, 60 hours, 80 hours and 100 hours later.

(2) Duration of reducing power: The duration of time during which the aqueous solutions prepared above maintain negative oxidation/reduction potential values was defined as duration of reducing power, and the duration from the time when the first firing product or the hydrogen anion-containing material was dissolved until when the oxidation/reduction potential value became 0 was measured.

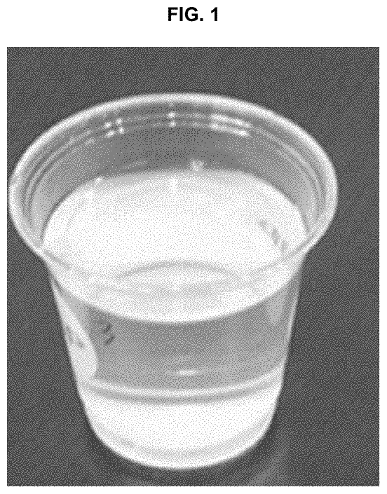

(3) Turbidity of aqueous solution: After observing the aqueous solution in which the first firing product or the hydrogen anion-containing material was dissolved with naked eyes, a turbid was marked as ‘turbid’ and a clear solution was marked as ‘clear’. As a representative example, the image of the aqueous solution of Example 1 is shown in FIG. 1.

Then, the anti-inflammatory activity of the anti-inflammatory composition was measured as follows.

A carbon dioxide (CO₂) incubator (Heracell 150i) was used for cell culturing. An ultrapure water system (Sartorius Arium 611VF) was used for preparation of ultrapure water (deionized water), and a high-pressure sterilizer (AC-14) was used for sterilization of experimental equipment.

The compositions prepared in Examples 1-6 were filtered through a 0.45-μm filter, and the filtrate was mixed with a culture medium per se, after being diluted 2-fold or after being diluted 4-fold, with a ratio of medium:sample=90:10 (i.e., the sample was finally diluted 10-fold, 20-fold or 40-fold).

The following experimental materials were used.
Raw 264.7 macrophages (TIB-71)
DMEM (+10% FBS, 1% antibiotics)
37° C. CO₂ incubator
PBS (phosphate-buffered saline) pH 7.4
LPS (lipopolysaccharide) stock solution (1 mg/mL)
Griess reagent system (Promega)
Cell counting kit-8 (Dojindo)
Cell culture flask (75 cm²), 24-well plate, 96-well plate

The experiment was performed as follows.

a. Raw 264.7 cells were cultured in DMEM (+10% FBS, 1% antibiotics).

b. The cultured cells were transferred to a 24-well plate with 2.5×105 cells/well and then cultured for 24 hours.

c. After removing the culture and adding 450 μL of a medium containing LPS (final concentration: 1 μg/mL) and 50 μL of a sample, the cells were cultured for 24 hours (i.e., the sample was finally diluted 10-fold).

d. After recovering the culture, nitrite content was measured on a 96-well plate using a Griess reagent system.

e. After the experiment was finished, the 24-well plate was subjected to CCK-8 assay after replacing with a fresh medium. After measuring cytotoxicity, only the sample with no toxicity [cell viability (% of control)≥80%] was subjected to anti-inflammatory activity evaluation.

### Test Example: Measurement of Cytotoxicity of Anti-Inflammatory Composition

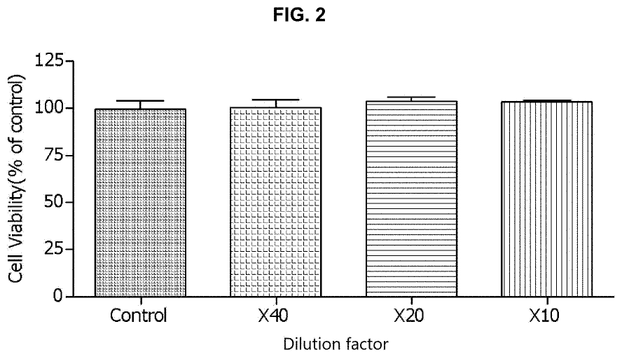

It was investigated whether the anti-inflammatory composition has cytotoxicity by treating Raw 264.7 cells with the anti-inflammatory composition according to the present disclosure and measuring cell viability. The result is shown in FIG. 2.

FIG. 2 shows average cell viability for the samples of Examples 1-6.

In FIG. 2, control refers to a non-treated control group, and the cells of each group were cultured for 24 hours after being treated with the sample at different concentrations. The height of each bar in FIG. 2 indicates mean±SD (n=6).

In FIG. 2, *, ** and *** mean *p<0.05, **p<0.01 and ***p<0.001, respectively, with respect to the control group.

From FIG. 2, it can be seen that the anti-inflammatory composition according to the present disclosure shows nearly the same cell viability as the control group irrespectively of the dilution factor (x40, x20, x10).

That is to say, it can be seen that the anti-inflammatory composition according to the present disclosure exhibits no cytotoxicity at all.

In addition, the anti-inflammatory activity of each sample was investigated as follows.

### Test Example: Investigation of Anti-Inflammatory Activity of Anti-Inflammatory Composition

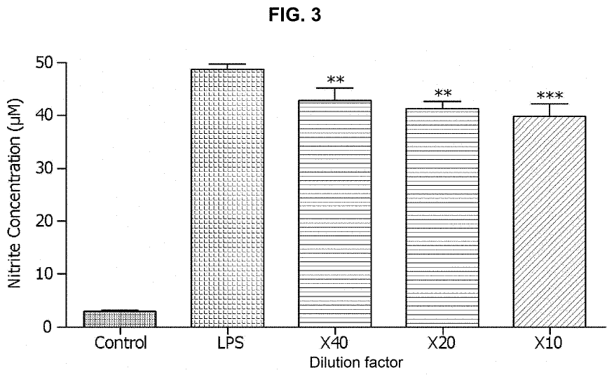

The anti-inflammatory activity of the anti-inflammatory composition was investigated by treating Raw 264.7 cells stimulated with LPS (lipopolysaccharides) with the anti-inflammatory composition according to the present disclosure and then measuring the production of nitrite (μM). The result is shown in FIG. 3.

LPS was used to induce inflammation. In FIG. 3, control refers to a control group not treated with LPS, and the cells of each group were cultured for 24 hours after being stimulated with LPS (1 μg/mL) and treated with the sample at different concentrations. The height of each bar in FIG. 3 indicates mean±SD (n=6).

In FIG. 3, *, ** and *** mean *p<0.05, **p<0.01 and ***p<0.001, respectively, with respect to the group treated with LPS only.

From FIG. 3, it can be seen that, as a result of evaluating the anti-inflammatory of the anti-inflammatory composition according to the present disclosure (NO assay), the production of the anti-inflammatory marker nitrite showed a distinct tendency of decreasing in a concentration-dependent manner, with 48.5 μM in the group treated with LPS only, 42.7 μM for the 40-fold diluted sample, 41.3 μM for the 20-fold diluted sample, and 39.6 μM for the 10-fold diluted sample.

That is to say, it was confirmed that the NO production decreased proportionally to the increased concentration (i.e., decreased dilution factor) of the anti-inflammatory composition of the present disclosure, suggesting that the anti-inflammatory composition according to the present disclosure exhibits anti-inflammatory activity in living cells.

1. An anti-inflammatory composition having hydrogen anions and prepared by a process comprising:
a first firing step of preparing a first firing product by firing an alkaline earth metal compound comprising one or more organic calcium carbonate selected from a group consisting of eggshell calcium, pearl calcium, shell calcium and seaweed calcium;
a second firing step of preparing a second firing product by firing an alkaline earth metal oxide comprising the first firing product under hydrogen atmosphere; and
a step of mixing the second firing product with an organic acid.

2. The anti-inflammatory composition having hydrogen anions according to claim 1, wherein, in the first firing step, the alkaline earth metal compound is fired at 300-1000° C. for 2-10 hours.

3. The anti-inflammatory composition having hydrogen anions according to claim 1, wherein the alkaline earth metal compound further comprises a beryllium compound, a magnesium compound, a barium compound or a mixture thereof.

4. The anti-inflammatory composition having hydrogen anions according to claim 1, wherein beryllium oxide, magnesium oxide, barium oxide or a mixture thereof is added in the second firing step.

5. The anti-inflammatory composition having hydrogen anions according to claim 1, wherein the alkaline earth metal oxide comprises 1-60 mol of beryllium oxide, magnesium oxide, barium oxide or a mixture thereof based on 100 mol of calcium oxide.

6. The anti-inflammatory composition having hydrogen anions according to claim 1, wherein, in the second firing step, the alkaline earth metal oxide is fired at 300-1000° C. for 2-10 hours under hydrogen atmosphere.

7. The anti-inflammatory composition having hydrogen anions according to claim 1, wherein the second firing product is mixed with 0.5-10 mol of an organic acid based on 100 mol of calcium ions comprised in the second firing product.

8. The anti-inflammatory composition having hydrogen anions according to claim 1, wherein the anti-inflammatory composition has a pH of 6-12 immediately after being dissolved in water; or has an oxidation/reduction potential of −0.1 mV to −900 mV or has a reducing power duration of 1-150 hours immediately after being dissolved in water.
